(19) United States
(12) Patent Application Publication
(10) Pub. No.: US 2005/0019210 A1
(43) Pub. Date: Jan. 27, 2005

(54) Parametric decontamination of bio-contaminated facities using chlorine dioxide gas

(76) Inventors: Aron A Rosenblatt, New York, NY (US); Thomas E. McWhorter, Allentown, PA (US); David Hirsch Rosenblatt, Baltimore, MD (US)

Correspondence Address: RATNERPRESTIA, P O BOX 980, VALLEY FORGE, PA, 19482-0980, US

(21) Appl. No.: 10/494,219

(22) PCT Filed: Nov. 4, 2002
(86) PCT No.: PCT/US02/35523

Related U.S. Application Data
(60) Provisional application No. 60/338,993, filed on Nov. 5, 2001.

(51) Int. Cl. A61L 2/20

(52) U.S. Cl. 422/37; 422/33

(57) ABSTRACT

Method for decontaminating structures by sealing the structure and introducing a chlorine dioxide gas/diluent gas mixture into and circulating through the structure to kill bio-contaminants in the structure.

### BACKGROUND OF THE INVENTION

From time to time, buildings have become contaminated by biological pathogens and require decontamination. Decontaminating methods include the use of foams and liquid anti-microbial agents, such as bleach, to disinfect surfaces. For decontamination of facilities that may have been subject to pathogens that can aerosolize, such as the finely divided Anthrax spores employed by bio-terrorists, it is advantageous to employ a decontaminating gas. Such gases may include, for example, chlorine dioxide. Gas molecules can decontaminate any aerosolized, airborne pathogens, and also can diffuse thoroughly through all the cracks and crevices in a facility and reach any surface that might have been reached by the target pathogen(s).

Chlorine dioxide gas is well known to kill resistant pathogenic organisms, such as bacillus subtilus v. niger, that are commonly used surrogates for pathogenic organisms, such as Anthrax spores. The extent of microbial kill by chlorine dioxide gas, as with other chemosterilants, is a function of several factors, including contact time, humidity and gas concentration.

At a partial-pressure gas concentration greater than about 76 mm Hg, chlorine dioxide gas may decompose explosively to chlorine and oxygen at standard temperature and pressure (STP), 76 mm Hg is about 10% in air. (10% ClO₂=100,000 ppm by volume.)

Chlorine dioxide is an acute irritant, which can cause lung damage and other adverse health effects. The acute toxicity of chlorine dioxide gas is a function of concentration. The 8-hour TLV for chlorine dioxide is 0.1 ppm; the 15 minute STEL is 0.3 ppm.

Chlorine dioxide is a strong oxidant. It bleaches certain dyes and pigments, and it reacts with some polymeric materials in ways that may cause functional or aesthetic damage. Unwanted interactions with some materials by chlorine dioxide gas are a function of concentration and time of exposure. Additionally, chlorine dioxide generated by some methods, such as acidification of sodium chlorite solution with HCl or reaction of sodium chlorite solution with hypochlorous acid, may contain chlorine as an impurity. The solutions used in such methods also may be highly acidic. If the means of generating chlorine dioxide gas involves starting with a solution-based method and “sparging” the gas product from the liquid, acid vapor as well as chlorine gas may be contained in the chlorine dioxide product. Chlorine, especially in the presence of humidity, is highly corrosive to metals and incompatible with many non-metallic materials. Chlorine gas also interferes, giving “false positives”, with many analytical techniques used to measure chlorine dioxide gas. Acid vapors are also corrosive. Substantially chlorine-free chlorine dioxide can be produced by certain methods, such as in the Gas:Solid method, or chlorine can be selectively removed from the chlorine dioxide by any of several methods, prior to use of the chlorine dioxide for decontamination.

In deploying chlorine dioxide gas for building decontamination, it is essential to use a sufficient amount of gas for a sufficient length of time to assure that pathogens have been killed. In addition, it is advantageous to mitigate the possibility of a chlorine dioxide gas explosion, to minimize the chances for personal exposure to toxic concentrations of chlorine dioxide gas, and to minimize deleterious effects on materials within the facility being treated. It is also advantageous to get the facility back in operation as quickly as possible.

Chlorine dioxide is subject to photolytic decomposition, under which it breaks down to chlorine and oxygen. In order to preserve the decontaminating ability of the chlorine dioxide gas, and to avoid the deleterious effects of chlorine gas, it is therefore necessary to protect chlorine dioxide from light, especially from ultra-violet light.

Gas sterilization is well known in the medical device and pharmaceutical industries where it has been employed to treat packaged medical devices and, to a limited extent, isolators (i.e., “clean rooms). Microbial inactivation with gaseous chemosterilants is a function of several parameters, including gas concentration, time, temperature and relative humidity. It is a preferred practice in the medical device manufacturing industry to develop knowledge of and document the set of inter-related parameters required to achieve a desired level of “kill” for a particular target organism, and to then assume that a device has been sterilized if it can be shown that the device has been subjected to conditions which at least meet said parameters. This allows for a quantitative, measurable, documentable basis for the device to be released as “sterile”, without relying on the indirect, somewhat-qualitative culturing and testing of biological indicators or by the collection and incubation of “swipe samples” (e.g., by swabbing surfaces)

Typically, there is some tradeoff between critical gas-sterilization parameters of time, relative humidity, temperature and gas concentration, but the relationships are not necessarily linear. It is customary to use a non-pathogenic surrogate organism to model the expected behavior of a highly pathogenic organism. Bacillus subtilus v. niger is widely recognized as an appropriate surrogate for chemo-sterilization of resistant organisms, such as Anthrax, and have been used to develop and validate medical-sterilization regimes. Because medical devices are substantially contamination-free prior to sterilization, the standard for assuring sterility is a cycle that reliably achieves 6-logs of “kill”. However, a sterilant's ability to achieve a certain level of kill, e.g., 6 logs, does not necessarily mean that a higher concentration of a sterilizing agent, or its application for a longer period of time, will be able to achieve higher levels of kill.

When pathogens are intended for use as biological warfare agents (BWA), as in recent cases of mail-borne Anthrax, the pathogens may be specially-prepared (“weaponized”) so that they can aerosolize and be inhaled by victims. Weaponized spores, such as those that cause the particularly deadly “inhalation Anthrax”, have several distinguishing characteristics: (1) They are small—reportedly on the order of 1-3 microns in size. This facilitates their easy dispersion, and ready entry deep into victims' lungs. (2) The particles remain discreet, i.e., they don't “clump” together, and are able to be aerosolized; and (3) in at least some cases, there is a high concentration of spores per unit of material. The weaponized Anthrax in the well publicized mail contamination cases reportedly contained 10⁸-10¹² spores per gram.

The weaponizing process involves multiple steps, including drying and milling spores to the desired size. However, several factors, including the natural hygroscopicity of spores and electrostatic surface charges that may be associated with milling fine particles, may cause the finely milled spores to clump together. In order to keep weaponized spores finely divided and to prevent “clumping”, they may be treated in various ways. Such processes help prevent clumping and facilitate aerosolization. However, these procedures also make much more difficult the inactivation of the dry, fine-milled spores. Procedures that are sufficient to kill natural” spores are not necessarily effective against “weaponized” spores.

In a well-publicized plan to use chlorine dioxide gas to decontaminate a government office building contaminated with weaponized Anthrax spores that were released from an Anthrax-containing letter, a chlorine dioxide solution was created by a conventional sodium chlorite solution-based process, the “3-chemical method”. Hydrochloric acid, sodium hypochlorite solution and sodium chlorite solution were mixed together followed by “sparging” chlorine dioxide gas from the solution in a gas “stripper”. This sparged chlorine dioxide-containing gas was pumped into the heating, ventilating and air conditioning (HVAC) system of the building, in amounts that were believed to be sufficient to fill the building with chlorine dioxide gas at a target concentration of approximately 500 ppm, at a temperature of 75° F. and 75% relative humidity for approximately 18 hours. Reportedly, the building was “tented” to mitigate escape of chlorine dioxide fumes. After about 24 hours of chlorine dioxide residence, the gas was originally planned to be pumped out through a “scrubber” containing ascorbic acid, which is a well known dechlorinating agent that reduces chlorine dioxide to chloride ion. (The scrubber reportedly was not used.) Determination of the effectiveness of the procedure relied on the testing of standard b. subtilus biological indicators, i.e., spore strips, placed throughout the facility prior to decontamination. These standard bio-indicators reportedly contained 10⁶ “natural” organisms—sufficient to indicate a maximum 6-log spore reduction. These standard spore strips were not correlated with the harder-to-kill “weaponized” Anthrax which comprised the target bio-contaminant. Determination of the effectiveness of the procedure also relied on comprehensive “swipe” sampling. Reportedly, the chlorine dioxide atmosphere in the building contained substantial percent-quantities of chlorine gas. Chlorine dioxide gas concentrations were uneven throughout the facility, and target concentrations were not uniformly met. The entire procedure was repeated at least three times, over more than 9 months, at a cost that reportedly exceeded $45 million.

### BRIEF SUMMARY OF THE INVENTION

A goal of this invention is to provide a method for chlorine dioxide gas decontamination of bio-contaminated facilities, that uses high-purity chlorine dioxide gas in the quantity and for the time period sufficient to kill pathogenic organisms, especially “weaoponized” spores, while minimizing the amounts of corrosion, risk of chlorine dioxide explosion, and risk of personal exposure to chlorine dioxide. Another goal of this invention is to document that sterilization parameters (correlated to the target organism at an appropriate log kill) have been achieved, so that the facility can be confidently certified ready for re-occupancy as quickly as personal-safety considerations allow.

Therefore, in a primary aspect the present invention is a method for decontaminating interior surfaces as well as the contents of a structure, suspected to contain bio-contamination comprising the steps of: sealing the structure to make it substantially air tight; eliminating substantially all illumination inside the structure and light entering the structure from ambient surroundings; optimally, adding humidity to the interior environment of the structure, introducing a substantially chlorine free chlorine dioxide gas/diluent gas mixture into the structure, until a sterilizing concentration (correlated to the target pathogen) of chlorine dioxide is reached throughout the structure; and maintaining the chlorine dioxide concentration inside the structure for a time sufficient to kill the bio-contaminant.

In another aspect of the present invention is a method for decontaminating interior surface and contents of a building suspected to contain bio-contamination, comprising the steps of: sealing the building to become substantially air tight; eliminating substantially all illumination inside the building and light entering the building from ambient surroundings; creating a slight negative pressure in the building; introducing a substantially chlorine free chlorine dioxide gas/diluent gas mixture into the building until a sterilizing concentration of chlorine dioxide is reached throughout the building; and maintaining the chlorine dioxide concentration inside the building for a time sufficient to kill the bio-contamination.

### DETAILED DESCRIPTION OF THE INVENTION

### EXAMPLE

In a preferred embodiment of the invention, (1) a contaminated facility would be sealed so that it was substantially air tight and dark. (2) In the case of a building facility, the building's heating ventilating and air combusting (HVAC) system would be operated in a mode that created and maintained a slight negative pressure on the building's interior; this can be achieved by drawing a small portion of the circulating air from the HVAC system through a scrubber to remove the chlorine dioxide, and venting the scrubbed gas outside the building. The amount of gas vented must be sufficient to offset the amount of air pulled through leaks into the building by the slight negative pressure. (3) Humidity would be introduced into the building (e.g., via the HVAC system) and circulated throughout until a target relative humidity of e.g., at least 60% and preferably 80%, is reached. High-purity, substantially chlorine-free chlorine dioxide gas would then be introduced into the building's interior (e.g., via the HVAC system, fire-suppression system or other means) and circulated throughout the building until a target gas concentration (e.g., 1000 ppm) is reached. Fans could be used to force circulation into areas that do not receive good circulation from the gas-distribution (e.g., HVAC) system; (4) the gas concentration would be monitored by means of sensors deployed throughout the building, and (5) “make up” gas would be fed, as needed, to assure that decontaminating concentrations were maintained. (6) Temperature, time and relative humidity would also be monitored and adjusted, as necessary. (7) On documenting that the parameters necessary for disinfection have been reached throughout the facility, and without reliance solely on biological-indicator testing or “swipe sampling”, the building could be safely reoccupied. In certain situations, it may be beneficial to increase the humidity inside the building and sustain high levels of humidity for several hours prior to introduction of the chlorine dioxide gas. In such circumstances where humidification and chlorine dioxide introduction are accomplished in separate steps, it may be advantageous to apply high purity chlorine dioxide gas that contains less than 50% humidity. This would help avoid problems associated with condensation of water from the chlorine dioxide gas, which can adversely affect gas introduction and also cause collateral damage to the building and its contents.

While the present invention has been described in relation to decontamination of a building, it is applicable to any structure that can be sealed and subjected to a negative pressure such as airplanes, tanks, ships and other marine vessels, vans, tunnels, subway systems and the like. And, while the present invention has been described in relation to decontamination of biological warfare agents, such as “weaponized” Anthrax, it is applicable to any biological pathogens, e.g., toxic mold (Stachybotrys) in water-damaged buildings; Staphyllococcus in hospitals, that can contaminate the interior of substantially-sealable structures.

In order to decontaminate other structures the same process would be used. However, if the structure e.g. a tank did not have an HVAC system other means would be employed to create a slight negative pressure in the vessel to assure circulation of the sterilizing gas to all parts of the vessel. A small inlet port or valve communicating with the ambient non-contaminated atmosphere and the source of sterilizing gas could be used in conjunction with an outlet point or valve connected to a vacuum pump to facilitate circulation of the sterilizing gas. The sterilizing gas removed by the vacuum pump would be passed through a scrubber to remove any chlorine dioxide prior to venting to the ambient atmosphere.

Having thus described our invention, what is desired to be secured by Letters Patent of the United States is set forth in the appended claims.

1. A method for decontaminating interior surfaces and contents of a structure suspected to contain bio-contamination, comprising the steps of:
sealing said structure to become substantially air tight;
eliminating substantially all illumination inside said structure and light entering said structure from ambient surroundings;
introducing a substantially chlorine-free chlorine dioxide gas diluent gas mixture into said structure, until a sterilizing concentration of chlorine dioxide is reached throughout said structure; and
maintaining said chlorine dioxide concentration inside said structure for a time sufficient to kill said bio-contamination.

2. A method according to claim 1 including the step of controlling sterilant-gas concentration, relative humidity and time to achieve greater than 6 logs of spore inactivation, of “weaponized” spores, or non-pathogenic, similarly-prepared surrogates of said spores.

3. A method according to claim 2 including the step of controlling sterilant gas concentration, relative humidity and time to achieve greater than 8 logs of inactivation of said spores.

4. A method according to claim 1 including the step of using chlorine dioxide gas containing less than 0.1% chlorine gas contaminant.

5. A method according to claim 1 including the step of producing said chlorine dioxide gas by one of, reacting dilute chlorine gas and an excess of solid sodium chlorite, reacting atomized sodium chlorite solution with chlorine gas, reacting sodium chlorite solution and an inorganic acid such as HCl, reacting sodium chlorite solution and hypochlorous acid, electrolysis of sodium chlorite solution, ultra-violet irradiation of sodium chlorite solution, acidification of sodium chlorate solution, or reduction of sodium chlorate solution; collecting said chlorine dioxide gas using an inert sweeping gas or gas-permeable membrane; and, scrubbing said chlorine dioxide/inert gas mixture of excess chlorine to produce a mixture of chlorine dioxide and inert gas substantially free of chlorine.

6. A method according to claim 1 including the step of maintaining said chlorine dioxide concentration from 500 to 10,000 ppm.

7. A method according to claim 1 including the step of monitoring atmosphere inside said structure using one or more analytical devices, whereby results from said analytical devices can be used to maintain a control device that maintains the required chlorine dioxide concentration inside said structure.

8. A method according to claim 1 including the step of maintaining relative humidity within said structure at a level of at least about 60%.

9. A method according to claim 1 including the step of maintaining an interior temperature in said structure of about 60° F. or higher.

10. A method according to claim 1 including the step of maintaining chlorine dioxide concentration, temperature, relative humidity, and time of exposure to chlorine dioxide at the required conditions within said structure until decontamination is complete.

11. A method according to claim 1 including the step of humidifying the air inside said structure to at least 60% relative humidity prior to introduction of the chlorine dioxide gas.

12. A method for decontaminating interior surface and contents of a building suspected to contain bio-contamination, comprising the step of:
sealing said building to become substantially air tight;
eliminating substantially all illumination inside said building and light entering said building from ambient surroundings;
creating a slight negative pressure in said building;
introducing a substantially chlorine-free chlorine dioxide gas/diluent gas mixture into said building until a sterilizing concentration of chlorine dioxide is reached throughout said building; and
maintaining said chlorine dioxide concentration inside said building for a time sufficient to kill said bio-contamination.

13. A method according to claim 12 including the step of controlling sterilant-gas concentration, relative humidity and time to achieve greater than 6 logs of spore inactivation, of “weaponized” spores, or non-pathogenic, similarly-prepared surrogates of said spores.

14. A method according to claim 12 including the step of controlling sterilant gas concentration, relative humidity and time to achieve greater than 8 logs of inactivation of said spores.

15. A method according to claim 12 including the step of maintaining said building under a slight negative pressure by continuously withdrawing a small portion of gaseous atmosphere from said building through a scrubber to remove chlorine dioxide from said withdrawn gaseous atmosphere and venting an amount of scrubbed gas sufficient to offset an amount of ambient atmosphere entering said building via any leaks in said building.

16. A method according to claim 12 including the step of using chlorine dioxide gas containing less than 0.1% chlorine gas contaminant.

17. A method according to claim 12 including the step of producing said chlorine dioxide gas by one of, reacting dilute chlorine gas and an excess of solid sodium chlorite, reacting atomized sodium chlorite solution with chlorine gas, reacting sodium chlorite solution and an inorganic acid such as HCl, reacting sodium chlorite solution and hypochlorous acid, electrolysis of sodium chlorite solution, ultra-violet irradiation of sodium chlorite solution; acidification of sodium chlorate solution, or reduction of sodium chlorate solution; collecting said chlorine dioxide gas using an inert sweeping gas or gas permeable membrane; and, scrubbing said chlorine dioxide/inert gas mixture of excess chlorine to produce a mixture of chlorine dioxide and inert gas substantially free of chlorine.

18. A method according to claim 12 including the step of maintaining interior portions of said building at a negative pressure of between 0 and 0.1 inches H₂0.

19. A method according to claim 12 including the step of maintaining said chlorine dioxide concentration from 500 to 10,000 ppm.

20. A method according to claim 12 including the step of monitoring atmosphere inside said building using one or more analytical devices, whereby results from said analytical devices can be used to maintain a control device that maintains the required chlorine dioxide concentration inside said building.

21. A method according to claim 12 including the step of maintaining relative humidity within said building at a level of at least about 60%.

22. A method according to claim 12 including the step of maintaining an interior temperature in said building of about 60° F. or higher.

23. A method according to claim 12 including the step of using materials impermeable to gas and bio-contaminants to seal portals and other openings in said building.

24. A method according to claim 12 including the step of humidifying the air inside said building to at least 60% relative humidity prior to introduction of the chlorine dioxide gas.
